(19) United States
(12) Patent Application Publication
(10) Pub. No.: US 2024/0001416 A1
(43) Pub. Date: Jan. 4, 2024

(54) SYSTEM FOR DETERMINING DISPOSAL OF SURGICAL INSTRUMENT AND RELATED METHODS

(71) Applicant: Cilag GmbH International, Zug (CH)

(72) Inventors: Frederick E. Shelton, IV, Hillsboro, OH (US); Jacqueline C. Aronhalt, Loveland, OH (US); Matthew D. Cowperthwait, Cincinnati, OH (US); Shane R. Adams, Lebanon, OH (US); Taylor W. Aronhalt, Loveland, OH (US); Joseph D. Paulowski, Butler, KY (US); Cesar Perez, Roseville, CA (US)

(21) Appl. No.: 17/854,110

(22) Filed: Jun. 30, 2022

(51) Int. Cl. B09B 3/00; A61B 17/32; A61B 90/90; G09B 19/00

(52) U.S. Cl. CPC B09B 3/00; A61B 17/320068; A61B 90/90; G09B 19/003; B09B 2101/65

(57) ABSTRACT

A method of determining a disposal methodology of a surgical kit includes determining a geographical location in which the surgical kit is being used, generating a location data based on the geographical location, and providing the location data set to a resource device. The method further includes receiving the disposal methodology from the resource device based on the location data set, then displaying a set of instruction based on the disposal methodology received from the resource device.

### BACKGROUND

A variety of ultrasonic surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. Examples of ultrasonic surgical instruments and related concepts are disclosed in U.S. Pub. No. 2006/0079874, entitled “Tissue Pad for Use with an Ultrasonic Surgical Instrument,” published Apr. 13, 2006, now abandoned, the disclosure of which is incorporated by reference herein, in its entirety; U.S. Pub. No. 2007/0191713, entitled “Ultrasonic Device for Cutting and Coagulating,” published Aug. 16, 2007, now abandoned, the disclosure of which is incorporated by reference herein, in its entirety; and U.S. Pub. No. 2008/0200940, entitled “Ultrasonic Device for Cutting and Coagulating,” published Aug. 21, 2008, now abandoned, the disclosure of which is incorporated by reference herein, in its entirety.

Some instruments are operable to seal tissue by applying radiofrequency (RF) electrosurgical energy to the tissue. Examples of such devices and related concepts are disclosed in U.S. Pat. No. 7,354,440, entitled “Electrosurgical Instrument and Method of Use,” issued Apr. 8, 2008, the disclosure of which is incorporated by reference herein, in its entirety; U.S. Pat. No. 7,381,209, entitled “Electrosurgical Instrument,” issued Jun. 3, 2008, the disclosure of which is incorporated by reference herein, in its entirety.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein, in its entirety; U.S. Pat. No. 8,663,220, entitled “Ultrasonic Electrosurgical Instruments,” issued Mar. 4, 2014, the disclosure of which is incorporated by reference herein, in its entirety; U.S. Pat. No. 10,835,307, entitled “Modular Battery Powered Handheld Surgical Instrument Containing Elongated Multi-Layered Shaft,” issued Nov. 17, 2020, the disclosure of which is incorporated by reference herein, in its entirety; and U.S. Pat. No. 11,229,471, entitled “Modular Battery Powered Handheld Surgical Instrument with Selective Application of Energy Based on Tissue Characterization,” issued Jan. 25, 2022, the disclosure of which is incorporated by reference herein, in its entirety.

In some scenarios, it may be preferable to have surgical instruments grasped and manipulated directly by the hand or hands of one or more human operators. In addition, or as an alternative, it may be preferable to have surgical instruments controlled via a robotic surgical system. Examples of robotic surgical systems and associated instrumentation are disclosed in U.S. Pat. No. 10,624,709, entitled “Robotic Surgical Tool with Manual Release Lever,” published on May 2, 2019, the disclosure of which is incorporated by reference herein, in its entirety; U.S. Pat. No. 9,314,308, entitled “Robotic Ultrasonic Surgical Device With Articulating End Effector,” issued on Apr. 19, 2016, the disclosure of which is incorporated by reference herein, in its entirety; U.S. Pat. No. 9,125,662, entitled “Multi-Axis Articulating and Rotating Surgical Tools,” issued Sep. 8, 2015, the disclosure of which is incorporated by reference herein, in its entirety; U.S. Pat. No. 8,820,605, entitled “Robotically-Controlled Surgical Instruments,” issued Sep. 2, 2014, the disclosure of which is incorporated by reference herein, in its entirety; U.S. Pub. No. 2019/0201077, entitled “Interruption of Energy Due to Inadvertent Capacitive Coupling,” published Jul. 4, 2019, the disclosure of which is incorporated by reference herein, in its entirety; U.S. Pub. No. 2012/0292367, entitled “Robotically-Controlled End Effector,” published on Nov. 11, 2012, now abandoned, the disclosure of which is incorporated by reference herein, in its entirety; and U.S. patent application Ser. No. 16/556,661, entitled “Ultrasonic Surgical Instrument with a Multi-Planar Articulating Shaft Assembly,” filed on Aug. 30, 2019, the disclosure of which is incorporated by reference herein, in its entirety.

Such instruments and robotic surgical systems may be further be incorporated into a surgical system for performing procedures in a surgical environment, such as surgical operating theaters or rooms in a healthcare facility. A sterile field is typically created around the patient and may include properly attired, scrubbed healthcare professions as well as desired furniture and/or fixtures. Examples of such surgical systems and associated features are disclosed in U.S. Pat. Pub. No. 2019/0201046, entitled “Method for Controlling Smart Energy Devices,” published on Jul. 4, 2019, the disclosure of which is incorporated by reference herein, in its entirety; U.S. Pat. Pub. No. 2019/0201080, entitled “Ultrasonic Energy Device Which Varies Pressure Applied by Clamp Arm to Provide Threshold Control Pressure at a Cut Progression Location,” published on Jul. 4, 2019, the disclosure of which is incorporated by reference herein, in its entirety; U.S. Pat. Pub. No. 2019/0201091, entitled “Radio Frequency Energy Device for Delivering Combined Electrical Signals,” published Jul. 4, 2019, the disclosure of which is incorporated by reference herein, in its entirety; U.S. Pat. Pub. No. 2019/0274717, entitled “Methods for Controlling Temperature in Ultrasonic Device,” published Sep. 12, 2019, the disclosure of which is incorporated by reference herein, in its entirety; and U.S. Pat. Pub. No. 2019/0207857, entitled “Surgical Network Determination of Prioritization of Communication, Interaction, or Processing Based on System or Device Needs,” published Jul. 4, 2019, the disclosure of which is incorporated by reference herein, in its entirety.

While several surgical instruments and systems have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a block diagram of an example a computer-implemented interactive surgical system;

FIG. 2 depicts a top schematic view of an example of a surgical system for performing a surgical procedure in an operating room of a healthcare facility;

FIG. 3 depicts a side schematic view of an example of a surgical hub of the surgical system of FIG. 2;

FIG. 4 depicts a perspective view of a combination generator module with bipolar, ultrasonic, and monopolar contacts of the surgical system of FIG. 2;

FIG. 5 depicts a side schematic view of an exemplary generator and various examples of surgical instruments for use with the surgical system of FIG. 2;

FIG. 6A depicts a schematic view of a surgical theater, where a surgical kit is located within a non-sterile entry room;

FIG. 6B depicts a schematic view of the surgical theater of FIG. 6A, where the surgical kit is transported into the sterile field of the surgical theater;

FIG. 6C depicts a schematic view of the surgical theater of FIG. 6A, where the surgical kit is divided into assembled surgical subcomponents, non-surgical subcomponents, and a plurality of disposal bags;

FIG. 6D depicts a schematic view of the surgical theater of FIG. 6A, where the surgical subcomponents and the non-surgical subcomponents are broken down into post-surgery components organized into categories;

FIG. 6E depicts a schematic view of the surgical theater of FIG. 6A, where the post-surgery components that are organized into categories and placed within a respective disposal bag of the surgical kit of FIG. 6A;

FIG. 6F depicts a schematic view of the surgical theater of FIG. 6A, where the post-surgery components and the disposal bags are transported into a non-sterile exit room;

FIG. 7 depicts a schematic view of an exemplary surgical instrument and a computational device;

FIG. 8 depicts a flowchart of a method for determining disposal instructions for an energized surgical instrument recently used in a surgical procedure;

FIG. 9A depicts a schematic view of a generator having a longer power cord and a surgical instrument having a shorter power cord, wherein the longer power cord is contained within a storage bin of the generator;

FIG. 9B depicts a schematic view of the generator and the surgical instrument of FIG. 9A, where the longer power cord extends away from the storage bin of the generator; and

FIG. 9C depicts a schematic view of the generator and the surgical instrument of FIG. 9A, wherein the longer power cord and the shorter power cord are coupled together.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. In addition, the terms “upper,” “lower,” “top,” “bottom,” “above,” and “below,” are used with respect to the examples and associated figures and are not intended to unnecessarily limit the invention described herein.

### I. Example of a Surgical System

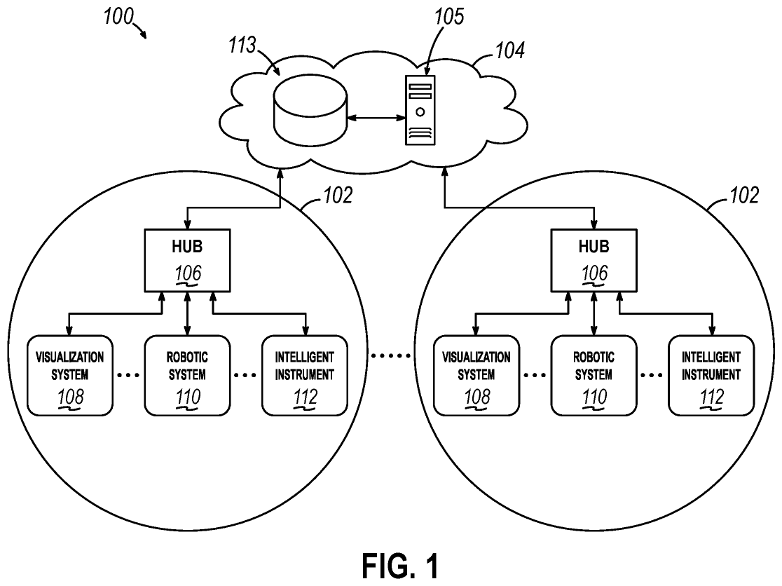

With respect to FIG. 1, a computer-implemented interactive surgical system (100) includes one or more surgical systems (102) and a cloud-based system (e.g., a cloud (104) that may include a remote server (113) coupled to a storage device (105)). Each surgical system (102) of the present example includes at least one surgical hub (106) in communication with cloud (104) that may include a remote server (113). In one example, as illustrated in FIG. 1, surgical system (102) includes a visualization system (108), a robotic system (110), and a handheld intelligent surgical instrument (112), which are configured to communicate with one another and/or hub (106). In some aspects, a surgical system (102) may include an M number of hubs (106), an N number of visualization systems (108), an O number of robotic systems (110), and a P number of handheld intelligent surgical instruments (112), where M, N, O, and P are integers greater than or equal to one. In any case, any suitable combination of features provided below may be incorporated into an exemplary surgical system, such as surgical system (100), and used in the surgical theater in order to perform a desired surgical procedure as would be apparent to one skilled in the art in view of the teachings herein.

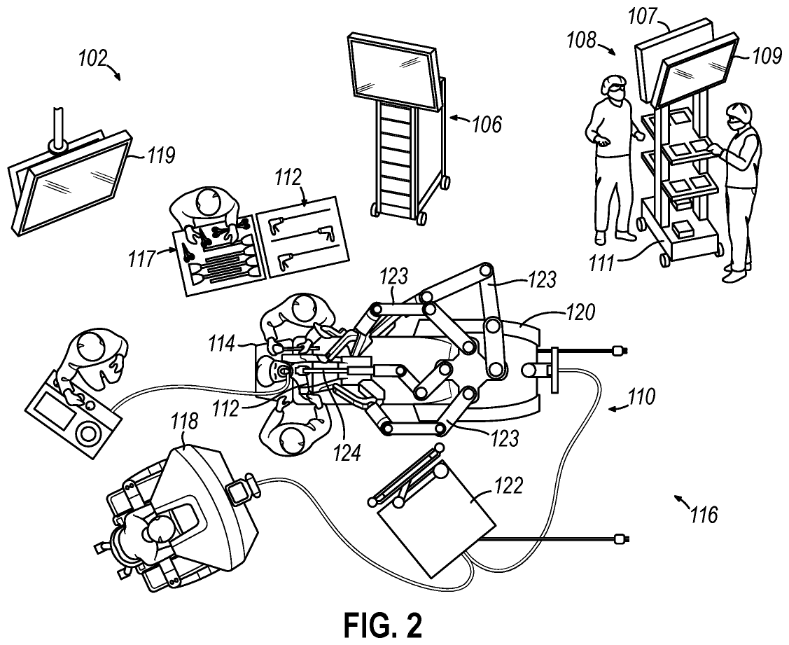

FIG. 2 depicts an example of a surgical system (102) being used to perform a surgical procedure on a patient who is lying down on an operating table (114) in a surgical operating room (116). A robotic system (110) is used in the surgical procedure as a part of surgical system (102). Robotic system (110) includes a surgeon's console (118), a patient side cart (120) (surgical robot), and a surgical robotic hub (122). Patient side cart (120) can manipulate at least one removably coupled surgical tool (117) with any one of a plurality of surgical arms (123) through a minimally invasive incision in the body of the patient while the surgeon views the surgical site through console (118). An image of the surgical site can be obtained by a medical imaging device (124), which can be manipulated by patient side cart (120) to orient imaging device (124). Robotic hub (122) can be used to process the images of the surgical site for subsequent display to the surgeon through console (118).

Other types of robotic systems can be readily adapted for use with surgical system (102). Various examples of robotic systems and surgical tools that are suitable for use with the present disclosure are described in U.S. Provisional Patent Application Ser. No. 62/611,339, entitled “Robot Assisted Surgical Platform,” filed Dec. 28, 2017, the disclosure of which is herein incorporated by reference in its entirety.

Various examples of cloud-based analytics that are performed by cloud (104, and are suitable for use with the present disclosure, are described in U.S. Provisional Patent Application Ser. No. 62/611,340, entitled Cloud-Based Medical Analytics,” filed Dec. 28, 2017, the disclosure of which is herein incorporated by reference in its entirety.

In various aspects, imaging device (124) includes at least one image sensor and one or more optical components. Suitable image sensors include, but are not limited to, Charge-Coupled Device (CCD) sensors and Complementary Metal-Oxide Semiconductor (CMOS) sensors. In various aspects, imaging device (124) is configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope. Some aspects of spectral and multi-spectral imaging are described in greater detail under the heading “Advanced Imaging Acquisition Module” in U.S. Provisional Patent Application Ser. No. 62/611,341, entitled “Interactive Surgical Platform,” filed Dec. 28, 2017, the disclosure of which is herein incorporated by reference in its entirety.

Strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

In addition to the introduction of any features of surgical system (100), furniture, or fixtures into the sterile field requiring sterilization, additional complications may result from removal of these features from the sterile field, particularly when such features may have contacted, or presumed to have contacted, the patient, including any tissues and/or fluids associated with the surgical procedure. Such contamination of these features from the patient often requires special consideration during or after the surgical procedure, particularly when processing these features for disposal, reuse, or remanufacturing as desired. In one example, surgical system (100) and/or healthcare professionals associated with the surgical procedure may be specifically equipped to address such processing as discussed below in greater detail.

As illustrated in FIG. 2, a primary display (119) is positioned in the sterile field to be visible to an operator at operating table (114). In addition, a visualization tower (111) is positioned outside the sterile field. Visualization tower (111) includes a first non-sterile display (107) and a second non-sterile display (109), which face away from each other. Visualization system (108), guided by hub (106), is configured to utilize displays (107, 109, 119) to coordinate information flow to operators inside and outside the sterile field. For example, hub (106) may cause visualization system (108) to display a snapshot of a surgical site, as recorded by imaging device (124), on a non-sterile display (107) or (109), while maintaining a live feed of the surgical site on the primary display (119). The snapshot on non-sterile display (107) or display (109) can permit a non-sterile operator to perform a diagnostic step relevant to the surgical procedure, for example.

In one aspect, hub (106) is also configured to route a diagnostic input or feedback entered by a non-sterile operator at visualization tower (111) to primary display (119) within the sterile field, where it can be viewed by a sterile operator at the operating table. In one example, the input can be in the form of a modification to the snapshot displayed on non-sterile display (107) or display (109), which can be routed to primary display (119) by hub (106).

Referring to FIG. 2, a surgical instrument (112) is being used in the surgical procedure as part of surgical system (102). Hub (106) is also configured to coordinate information flow to a display of the surgical instrument (112) such as in, for example, U.S. Provisional Patent Application Ser. No. 62/611,341, entitled “Interactive Surgical Platform,” filed Dec. 28, 2017, the disclosure of which is herein incorporated by reference in its entirety. A diagnostic input or feedback entered by a non-sterile operator at visualization tower (111) can be routed by hub (106) to surgical instrument display (115) within the sterile field, where it can be viewed by the operator of surgical instrument (112). Example surgical instruments that are suitable for use with surgical system (102) are described under the heading “Surgical Instrument Hardware” and in U.S. Provisional Patent Application Ser. No. 62/611,341, entitled “Interactive Surgical Platform,” filed Dec. 28, 2017, the disclosure of which is herein incorporated by reference in its entirety, for example.

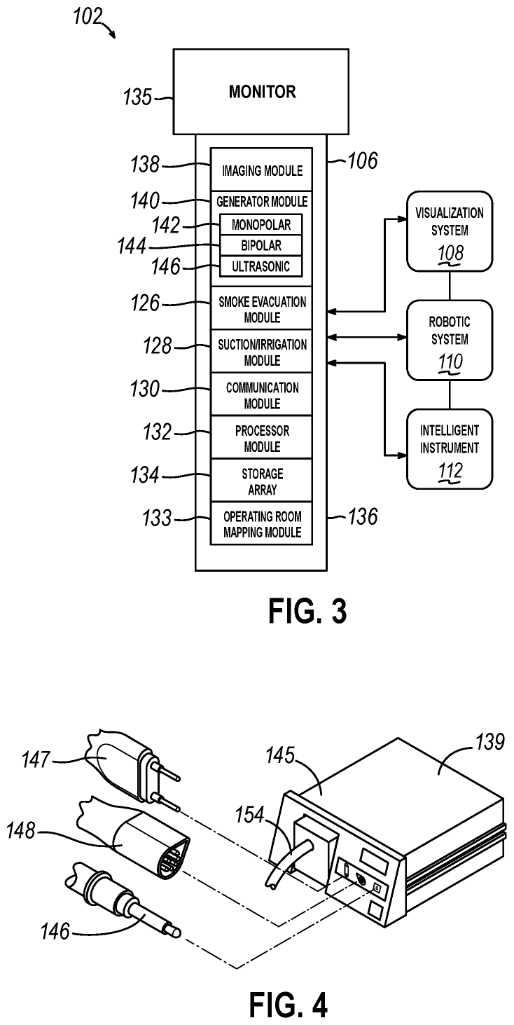

Referring now to FIG. 3, a hub (106) is depicted in communication with a visualization system (108), a robotic system (110), and a handheld intelligent surgical instrument (112). Hub (106) includes a hub display (135), an imaging module (138), a generator module (140), a communication module (130), a processor module (132), and a storage array (134). In certain aspects, as illustrated in FIG. 3, hub (106) further includes a smoke evacuation module (126), a suction/irrigation module (128), and/or an operating room mapping module (133).

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure (136) offers a unified environment for managing the power, data, and fluid lines, which reduces the frequency of entanglement between such lines.

Referring to FIGS. 3-4, aspects of the present disclosure are presented for a hub modular enclosure (136) that allows the modular integration of a generator module (140), a smoke evacuation module (126), and a suction/irrigation module (128). Hub modular enclosure (136) further facilitates interactive communication between modules (140, 126, 128). As shown in FIG. 4, generator module (140) can be a generator module with integrated monopolar, bipolar, and ultrasonic components supported in a single housing unit (139) slidably insertable into hub modular enclosure (136). As illustrated in FIG. 4, generator module (140) can be configured to connect to a monopolar device (146), a bipolar device (147), and an ultrasonic device (148). Alternatively, generator module (140) may comprise a series of monopolar, bipolar, and/or ultrasonic generator modules that interact through hub modular enclosure (136). Hub modular enclosure (136) can be configured to facilitate the insertion of multiple generators and interactive communication between the generators docked into the hub modular enclosure (136) so that the generators would act as a single generator.

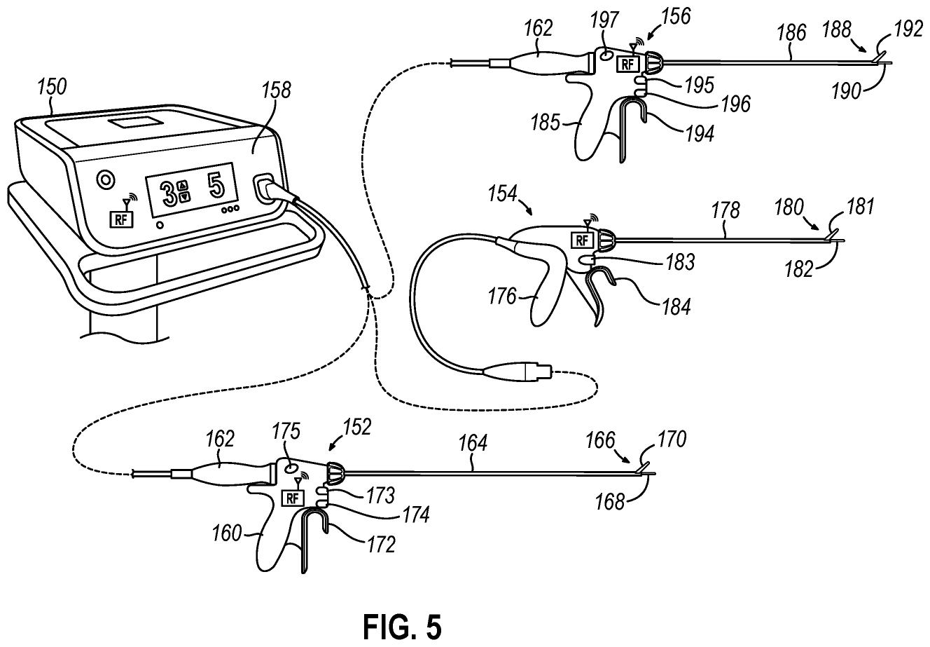

FIG. 5 illustrates one form of a generator (150) and various surgical instruments (152, 154, 156) usable therewith, where surgical instrument (152) is an ultrasonic surgical instrument (152), surgical instrument (154) is an RF electrosurgical instrument (154), and multifunction surgical instrument (156) is a combination ultrasonic/RF electrosurgical instrument (156). Generator (150) is configurable for use with a variety of surgical instruments. According to various forms, generator (150) may be configurable for use with different surgical instruments of different types including, for example, ultrasonic surgical instruments (152), RF electrosurgical instruments (154), and multifunction surgical instruments (156) that integrate RF and ultrasonic energies delivered simultaneously from generator (150). Although generator (150) of the present example in FIG. 5 is shown separate from surgical instruments (152, 154, 156), generator (150) may alternatively be formed integrally with any of surgical instruments (152, 154, 156) to form a unitary surgical system. Generator (150) comprises an input device (158) located on a front panel of generator (150) console. Input device (158) may comprise any suitable device that generates signals suitable for programming the operation of generator (150). Generator (150) may be configured for wired or wireless communication.

Generator (150) of the present example is configured to drive multiple surgical instruments (152, 154, 156). One example of such surgical instrument is ultrasonic surgical instrument (152) and comprises a handpiece (160), an ultrasonic transducer (162), a shaft assembly (164), and an end effector (166). End effector (166) includes an ultrasonic blade (168) acoustically coupled to ultrasonic transducer (162) and a clamp arm (170). Handpiece (160) has a trigger (172) to operate clamp arm (170) and a combination of toggle buttons (173, 174, 175) to energize and drive ultrasonic blade (168) or other function. Toggle buttons (173, 174, 175) can be configured to energize ultrasonic transducer (162) with generator (150).

Generator (150) also is configured to drive another example of surgical instrument (154). RF electrosurgical instrument (154) includes a handpiece (176), a shaft assembly (178), and an end effector (180). End effector (180) includes electrodes in clamp arms (181, 182) and return through an electrical conductor portion of shaft assembly (178). Electrodes are coupled to and energized by a bipolar energy source within generator (150). Handpiece (176) includes a trigger (183) to operate clamp arms (181, 182) and an energy button (184) to actuate an energy switch to energize electrodes in end effector (180).

Generator (150) also is configured to drive multifunction surgical instrument (156). Multifunction surgical instrument (156) includes a handpiece (185), a shaft assembly (186), and an end effector (188). End effector (188) has an ultrasonic blade (190) and a clamp arm (192). Ultrasonic blade (190) is acoustically coupled to ultrasonic transducer (162). Handpiece (185) has a trigger (194) to operate clamp arm (192) and a combination of toggle buttons (195, 196, 197) to energize and drive ultrasonic blade (190) or other function. Toggle buttons (195, 196, 197) can be configured to energize ultrasonic transducer (162) with generator (150) and energize ultrasonic blade (190) with a bipolar energy source also contained within generator (150). It will be appreciated that handpieces (160, 176, 185) may be replaced with a robotically controlled instrument for incorporating one or more aspects of surgical instruments (152, 154, 156). Accordingly, the term “handpiece” should not be limited to this context and to handheld use.

As used throughout this description, the term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some aspects they might not. The communication module may implement any of a number of wireless or wired communication standards or protocols, including but not limited to Wi-Fi (IEEE 802.11 family), WMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, Ethernet derivatives thereof, as well as any other wireless and wired protocols that are designated as 3G, 4G, 5G, and beyond. The computing module may include a plurality of communication modules. For instance, a first communication module may be dedicated to shorter range wireless communications such as Wi-Fi and Bluetooth and a second communication module may be dedicated to longer range wireless communications such as GPS, EDGE, GPRS, CDMA, WiMAX, LTE, Ev-DO, and others.

As used herein a processor or processing unit is an electronic circuit which performs operations on some external data source, usually memory or some other data stream. The term is used herein to refer to the central processor (central processing unit) in a system or computer systems (especially systems on a chip (SoCs)) that combine a number of specialized “processors.”

As used herein, a system on a chip or system on chip (SoC or SOC) is an integrated circuit (also known as an “IC” or “chip”) that integrates all components of a computer or other electronic systems. It may contain digital, analog, mixed-signal, and often radio-frequency functions, all on a single substrate. A SoC integrates a microcontroller (or microprocessor) with advanced peripherals like graphics processing unit (GPU), Wi-Fi module, or coprocessor. A SoC may or may not contain built-in memory.

As used herein, a microcontroller or controller is a system that integrates a microprocessor with peripheral circuits and memory. A microcontroller (or MCU for microcontroller unit) may be implemented as a small computer on a single integrated circuit. It may be similar to a SoC; an SoC may include a microcontroller as one of its components. A microcontroller may contain one or more core processing units (CPUs) along with memory and programmable input/output peripherals. Program memory in the form of Ferroelectric RAM, NOR flash or OTP ROM is also often included on chip, as well as a small amount of RAM. Microcontrollers may be employed for embedded applications, in contrast to the microprocessors used in personal computers or other general purpose applications consisting of various discrete chips.

As used herein, the term controller or microcontroller may be a stand-alone IC or chip device that interfaces with a peripheral device. This may be a link between two parts of a computer or a controller on an external device that manages the operation of (and connection with) that device. Modular devices include the modules (as described in connection with FIG. 3, for example) that are receivable within a surgical hub and the surgical devices or instruments that can be connected to the various modules in order to connect or pair with the corresponding surgical hub. The modular devices include, for example, intelligent surgical instruments, medical imaging devices, suction/irrigation devices, smoke evacuators, energy generators, ventilators, insufflators, and displays. The modular devices described herein can be controlled by control algorithms. The control algorithms can be executed on the modular device itself, on the surgical hub to which the particular modular device is paired, or on both the modular device and the surgical hub (e.g., via a distributed computing architecture). In some exemplifications, the modular devices' control algorithms control the devices based on data sensed by the modular device itself (i.e., by sensors in, on, or connected to the modular device). This data can be related to the patient being operated on (e.g., tissue properties or insufflation pressure) or the modular device itself (e.g., the rate at which a knife is being advanced, motor current, or energy levels). For example, a control algorithm for a surgical stapling and cutting instrument can control the rate at which the instrument's motor drives its knife through tissue according to resistance encountered by the knife as it advances.

### II. Exemplary Introduction, Assembly, Use, and Disposal of a Surgical Kit in a Surgical Theater

As mentioned above, objects intended to penetrate a sterile field of the surgical theater during a surgical procedure need to be suitably sterilized; while objects leaving the sterile field after a surgical procedure often require special consideration when processing for disposal, reuse, or remanufacturing. In some instances, surgical instrument/tool (112, 117, 152, 154, 156) may require at least some degree of assembly within the surgical theater prior to exemplary use in accordance with the description herein; and then require some degree of disassembly after exemplary use such that selective components may be disposed, reused, and/or remanufactured. For example, ultrasonic blade (168) may need to be suitably coupled to ultrasonic transducer (162) and/or shaft assembly (164) may need to be suitably coupled to handpiece (160) within the surgical theater prior to exemplary use of instrument (152). Additionally, after exemplary use, ultrasonic blade (168) may need to be decoupled from ultrasonic transducer (162) for suitable processing in accordance with the description herein.

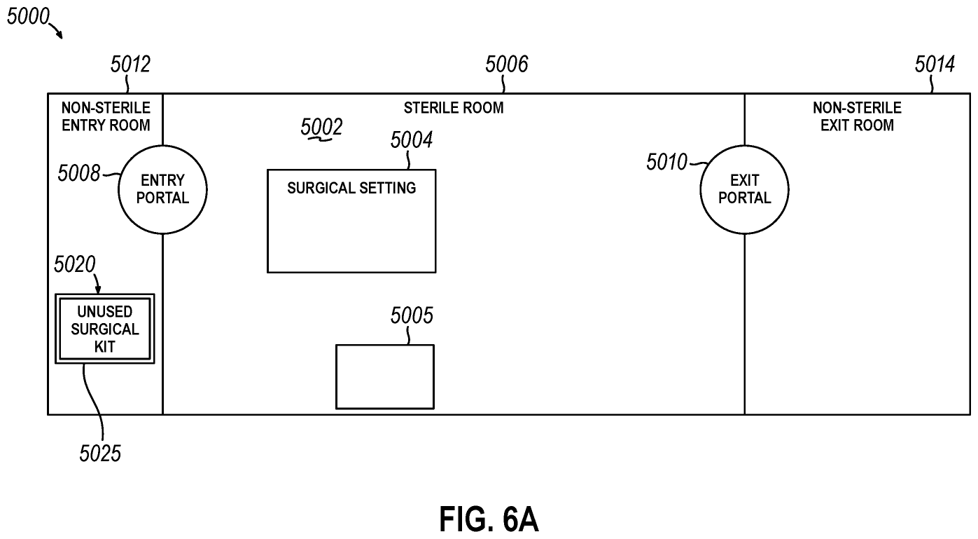

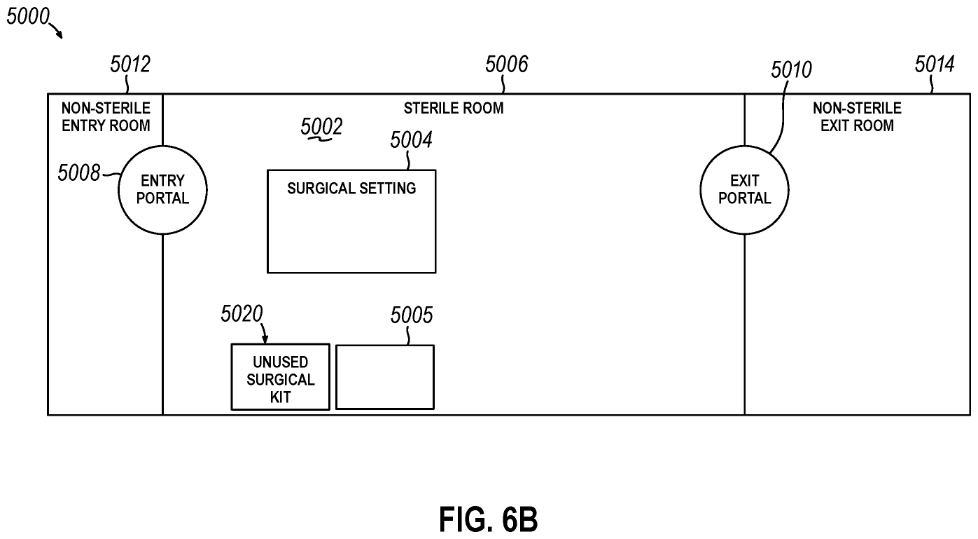

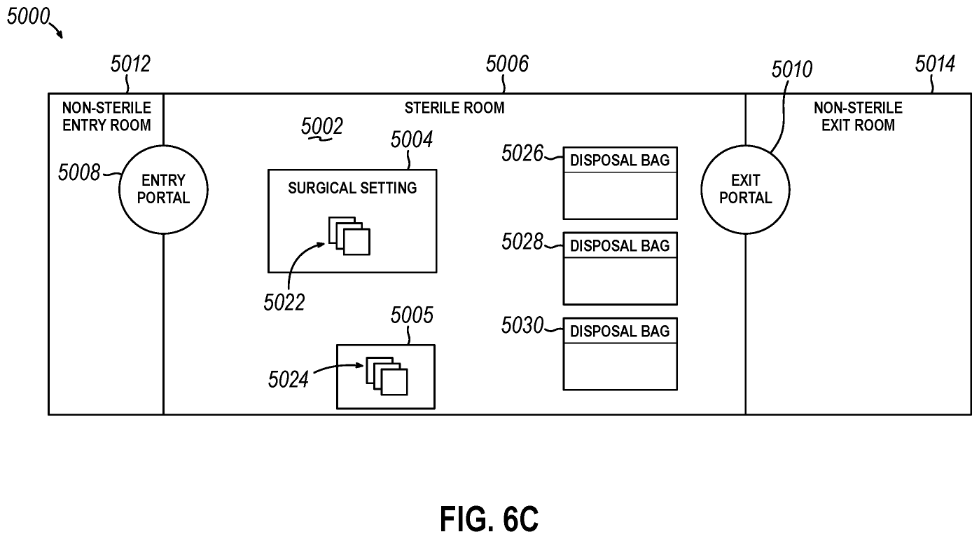

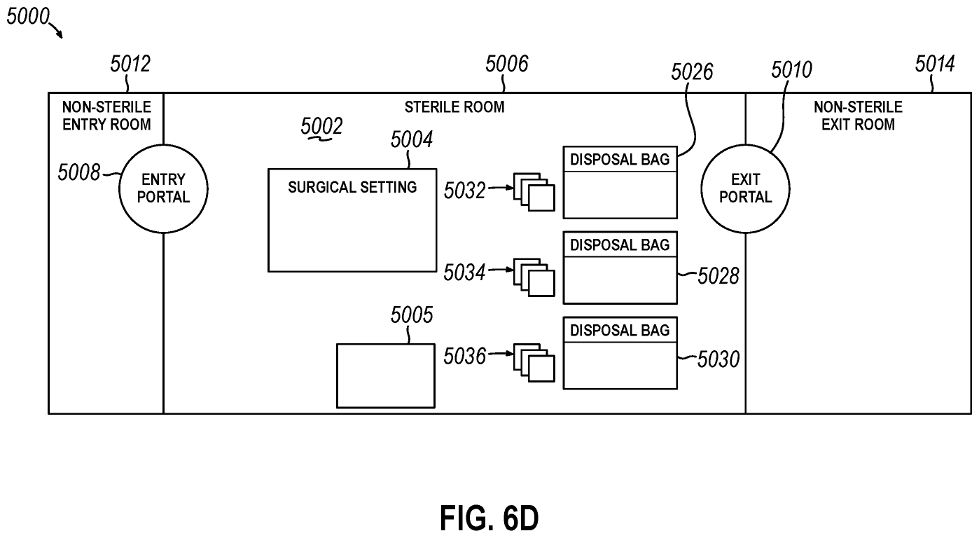

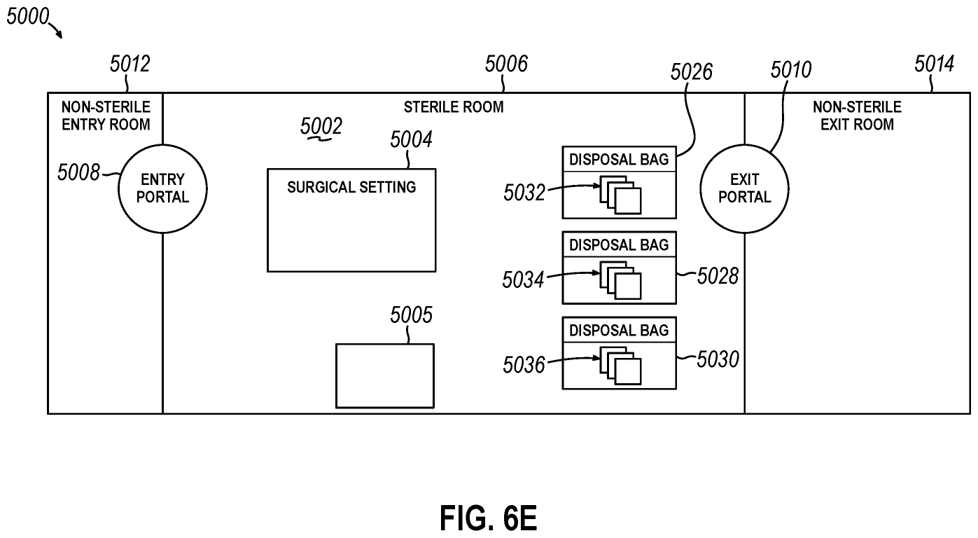

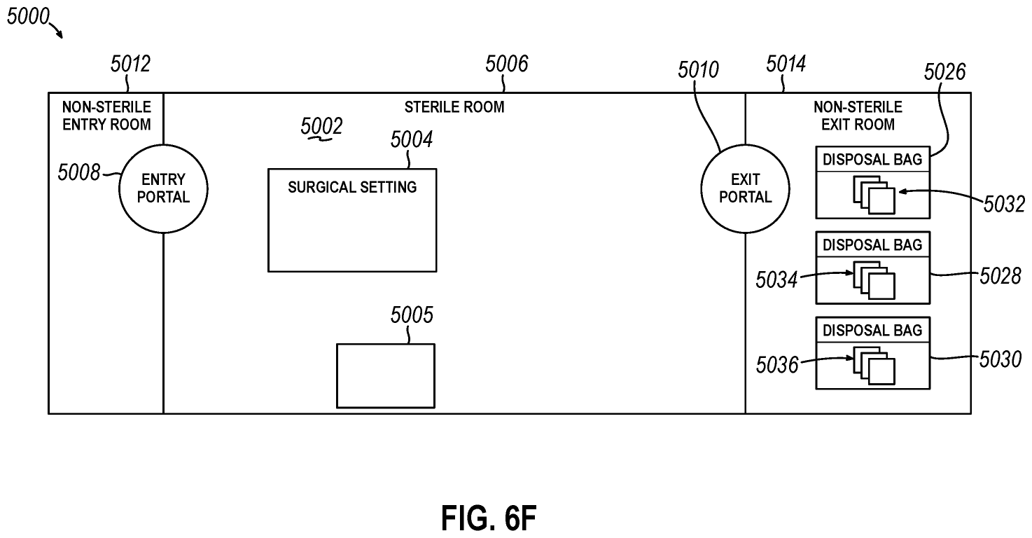

Therefore, in some instances, a surgical instrument/tool (112, 117, 152, 154, 156) may be introduced into the sterile field as a surgical kit with subcomponents requiring at least some degree of assembly prior to exemplary use. In addition to the subcomponents used to form surgical instrument/tool (112, 117, 152, 154, 156), such a surgical kit may contain the necessary tools for assembly, disassembly, and suitable processing for disposal, reuse, or remanufacturing of instrument/tool (112, 117, 152, 154, 156) and its subcomponents. FIGS. 6A-6F show an exemplary introduction, assembly, use, disassembly, and disposal of a surgical kit (5020).

Initially, as shown in FIG. 6A, prior to a surgical procedure, surgical kit (5020) may be contained within a sealed and sterilized interior of a storage bag (5025). Storage bag (5025) may have a non-sterile exterior such that non-sterilized persons may grasp the exterior of storage bag (5025) in order to transport surgical kit (5020) into non-sterile room (5012) in preparation for a surgical procedure as would be apparent to one skilled in the art in view of the teachings herein. Additionally, the sealed and sterilized nature of the interior of storage bag (5025) may help ensure surgical kit (5020) remains suitably sterilized while being transported within storage bag (5025).

In preparation for a surgical procedure, surgical kit (5020) may be transferred from non-sterile entry room (5012) into a sterile field (5006) of a surgical theater (5000), as shown in FIG. 6B. Surgical kit (5020) may be transferred into sterile field (5006) via a suitable entry portal (5008) as would be apparent to one skilled in the art in view of the teachings herein. During such a transfer, a person within non-sterile entry room (5012) may place kit (5020) and bag (5025) into entry portal (5008) and provide suitable access to, while maintaining the sterile nature of, the interior of bag (5025). Next, a sterilized person within sterile field (5006) may suitably access the sterilized interior of storage bag (5025) and remove surgical kit (5020) from storage bag (5025) such that kit (5020) is suitably transferred into both sterile room (5002) and sterile field (5006) as shown in FIG. 6B. Therefore, kit (5020) may remain suitably sterilized after being transferred from non-sterile entry room (5012) into surgical theater (5000).

Sterile room (5002) may be substantially similar to surgical operating room (116) described above. Sterile room (5002) includes a surgical setting (5004), which may be substantially similar to operating table (114) described above. Additionally, sterile room (5002) also includes an assembly station (5005), which may include a sterile table and/or any other suitable structures as would be apparent to one skilled in the art in view of the teachings herein.

Turning to FIG. 6C, once surgical kit (5020) is suitably transferred into sterile room (5002), the contents of surgical kit (5020) may be accessed and placed on assembly station (5005). In the current example, surgical kit (5020) includes surgical subcomponents (5022), non-surgical subcomponents (5024), and a plurality of disposal bags (5026, 5028, 5030). Surgical subcomponents (5022) are intended to be assembled in order to form a surgical instrument substantially similar to instrument/tool (112, 117, 152, 154, 156) described above. Non-surgical subcomponents (5024) may include portions of surgical kit (5020) that are not intended to be directly used in the surgical procedure, but for ancillary purposes to the surgical procedure. For example, non-surgical subcomponents (5024) may include tool(s) for assembling and disassembling surgical subcomponents (5022), a tray that may organize other components (5022, 5024, 5026, 5028, 5030), or other features as desired for exemplary use in accordance with the description herein. As will be described in greater detail below, disposal bags (5026, 5028, 5030) are configured to store respective post-surgery components (5032, 5034, 5036) (see FIG. 6D) such that post-surgery components (5032, 5034, 5036) may be removed from sterile room (5002) while being suitably sealed from exposure to a non-sterile environment (5012, 5014).

As also shown in FIG. 6C, after being suitably assembled (either pre-assembled, assembled by hand within sterile room (5002), and/or utilizing tools from non-surgical subcomponents (5024)), surgical subcomponents (5022) may be utilized in the surgical setting (5004) to perform a suitable surgical procedure on a patient as would be apparent to one skilled in the art in view of the teachings herein. Surgical subcomponents (5022) may form any suitable instrument/tool (112, 117, 152, 154, 156) described herein. Therefore, surgical subcomponents (5022) may be configured as a component of computer-implemented interactive surgical system (100). For example, in instances where surgical subcomponents (5022) form a removably coupled surgical tool (117) intended for use with patient side cart (120), surgical subcomponents (5022) may be used in conjunction with robotic system (110). As another example, in instances where surgical subcomponents (5022) form a handheld intelligent surgical instrument (112), surgical subcomponents (5022) may be handled by a surgeon and suitably coupled with hub (106) during exemplary use in accordance with the description herein.

Turning to FIG. 6D, after suitable use, surgical subcomponents (5022) may have been exposed to the patient such that subcomponents (5022) require special consideration. Therefore, surgical subcomponents (5022) and non-surgical subcomponents (5024) may be broken down and compartmentalized into post-surgery components (5032, 5034, 5036). Any suitable means of disassembling subcomponents (5022, 5024) may be utilized as would be apparent to one skilled in the art in view of the teachings herein. Post-surgery components (5032, 5034, 5036) may be categorized based on the type of post-surgery processing intended for each category. For example, broken down subcomponents (5022, 5024) intended to be reused or remanufactured may be sorted into a first category of post-surgery components (5032); while broken down subcomponents (5022, 5024) containing electrical components intended to be disposed of may be sorted into a second category of post-surgery components (5034); and broken down subcomponents (5022, 5024) that are intended to be disposed of, but do not contain electrical components, may be sorted into a third category of post-surgery components (5036). At the moment shown in FIG. 6D, certain disposable components may be further processed in preparation for being disposed of properly. For example, an internal power source (e.g., battery) utilized as a surgical subcomponent (5022) may be required to be fully discharged at this moment.

Next, as shown in FIG. 6E, while still in sterile room (5002), categorized post-surgery components (5032, 5034, 5036) may then be placed within a respective disposal bag (5026, 5028, 5030). Disposal bags (5026, 5028, 5030) may then be sealed such that post-surgery components (5032, 5034, 5036) are sealed from the external environment outside of respective bags (5026, 5028, 5030). Disposal bags (5026, 5028, 5030) may utilize any suitable sealing means as would be apparent to one skilled in the art in view of the teachings herein.

Next, as shown in FIG. 6D, post-surgery components (5032, 5034, 5036) and respective disposal bags (5026, 5028, 5030) may be transported out of room (5002) and sterile field (5006) into a non-sterile room (5014) via an exit portal (5010). Therefore, post-surgery components (5032, 5034, 5036) may then be transported to a suitable location for disposal, reprocessing, and/or remanufacturing while remaining sealed off from the external environment. In instances where components (5032, 5034, 5036) may have contacted the patient during a surgical procedure, such components (5032, 5034, 5036) will be inhibited from undesirable exposure in non-sterile rooms (5012, 5014).

While in the current example, a non-sterile entry room (5012) and a non-sterile exit room (5014) are used in conjunction with entry portal (5008) and exit portal (5010), this is merely optional. In some instances, surgical kit (5020) and post-surgery components (5032, 5034, 5036) are transported from/to the same non-sterile room (5012, 5014) and utilizing the same portal (5008, 5010).

While in the current example disposal bags (5026, 5028, 5030) are used, and suitably disposal structure configured to suitably support respective components (5032, 5034, 5036) while sealing such components (5032, 5034, 5036) from the external environment during transport may be used as would be apparent to one skilled in the art in view of the teachings herein.

### III. Exemplary Device and Method for Determining and Extracting Disposal Instructions for a Used Energized Surgical Instrument

As mentioned above, after suitable use of surgical subcomponents (5022), surgical subcomponents (5022) (e.g., the portions of surgical kit (5020) used to form an energized surgical instrument/tool (112, 117, 152, 154, 156)) may be broken down into post-surgery components (5032, 5034, 5036) for purposes of disposal, reprocessing, and/or remanufacturing. As one example, such post-surgery components (5032, 5034, 5036) may be categorized as components (5032) intended to be reused/remanufactured, electrical components (5034) intended for disposal, and non-electrical components (5036) intended for disposal.

In some instances, there may be various standards for classifying which used surgical subcomponents (5022) may be reusable/manufacturable and which surgical subcomponents (5022) are disposable. Additionally, there may be various standards for suitably processing/disposing such reusable, manufacturable, and disposable surgical subcomponents; either within the sterile environment as surgical subcomponents (5022) are originally being disassembled, or outside the sterile environment while surgical subcomponents (5022) are being suitably reprocessed, remanufactured, and/or disposed of as would be apparent to one skilled in the art in view of the teachings herein. The standards and/or suggested guidelines for classifying subcomponents (5022) as either reusable or disposable may vary between jurisdictions. Additionally, the reprocessing, remanufacturing, and/or disposal standards and/or suggested guidelines may also vary between jurisdictions. Such standards/guidelines for classifying, reprocessing, remanufacturing, disposal, etc., of used surgical subcomponents (5022) may be referred to as disposal methodology.

For example, in a first jurisdiction, an energized component (e.g., electrodes, transducers, ultrasonic blades, batteries, etc.) may only be certified for a first number of surgical uses (such as one surgical procedure); while in a second jurisdiction, that same energized component may be certified for reprocessing and/or remanufacturing for a different number of surgical uses (such as multiple surgical procedures). As another example, in a first jurisdiction, an electrical component may require being completely discharged before being transferred to a respective disposal bag (5026, 5028, 5030); while in a second jurisdiction, the same electrical component may not require being completely discharged before being transferred to a respective disposal bag (5026, 5028, 5030).

Therefore, it may be desirable to retrieve a set of disposal instructions for processing the components of a surgical kit (5020) after an exemplary surgical procedure in accordance with the description herein. Further, it may be desirable to retrieve a specific set of disposal instructions for a surgical kit (5020) based any number of suitable variables that would be apparent to one skilled in the art in view of the teachings herein. For example, such variables may include, but are not limited to, the geographic location of the surgical procedure, the use of other surgical products in conjunction with surgical kit (5020), and/or performance data of surgical subcomponents (5022).

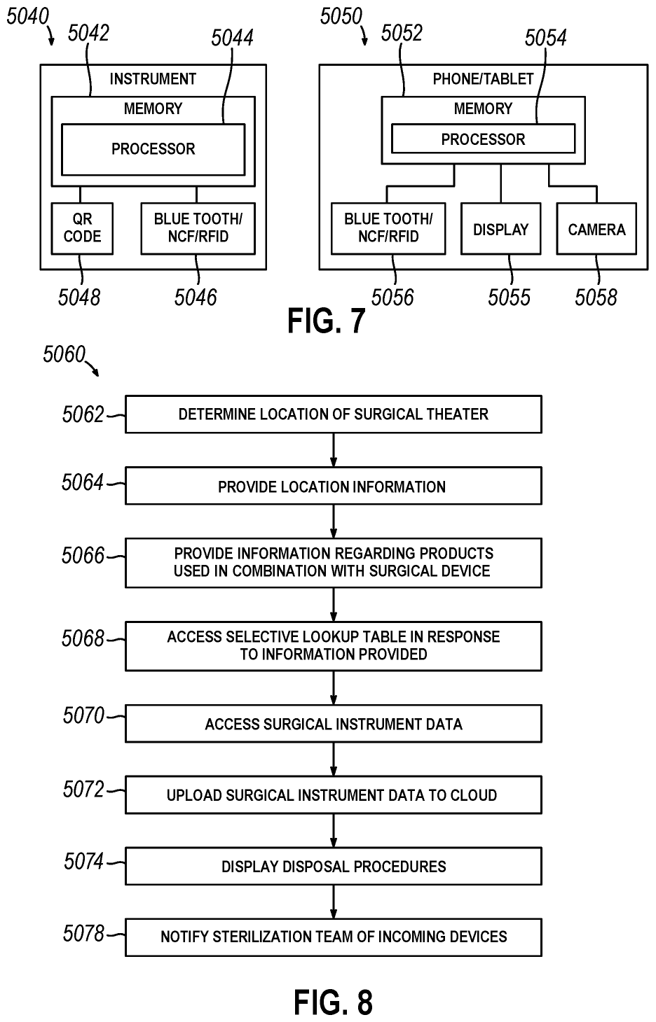

FIG. 7 shows an exemplary surgical instrument (5040) and an exemplary disposal assistance device (5050) that may be utilized in the determination procedure (5060) for disposal instructions shown in FIG. 8. It should be understood that while exemplary surgical instrument (5040) and disposal assistance device (5050) are utilized in the current example to perform determination procedure (5060) for disposal instructions as shown in FIG. 8, any suitable surgical instrument may be utilized, alone or in conjunction with any suitable disposal assistance device, as would be apparent to one skilled in the art in view of the teachings herein.

Surgical instrument (5040) may be assembled from a surgical kit (5020) such that surgical instrument (5040) is substantially similar to surgical subcomponents (5022) described above. Therefore, surgical instrument (5040) may be assembled within the surgical theater (5000) prior to being used in a surgical procedure, and then disassembled and categorized into various post-surgery components (5032, 5034, 5036). In some instances, such disassembly may occur within the surgical theater (500) after being used in a surgical procedure.

Surgical instrument (5040) may be substantially similar to any surgical instrument/tool described herein, including surgical instrument/tool (112, 117, 152, 154, 156) described above. Therefore, surgical instrument (5040) is configured to be used in conjunction with computer-implemented interactive surgical system (100) by coupling with suitable components of hub (106), such as generator module (140), communication module (130), storage array (134), etc. In some instances, surgical instrument (5040) may be handheld; while in other instances, surgical instrument (5040) may be configured to suitably couple with robotic system (110). In some instances, surgical instrument (5040) may be utilized without being connected to hub (1060), such that surgical instrument (5040) includes its own power source or such that surgical instrument (5040) is coupled to a generator other than one associated with hub (1060).

Surgical instrument (5040) includes a memory unit (5042) and a processing unit (5044); which may together function as a control unit for surgical instrument (5040). Memory unit (5042) may be operable to store various information related to the performance of surgical instrument (5040) during an exemplary surgical procedure in accordance with the description herein. Memory unit (5042) may also store data related to the various components used to form surgical instrument (5040). For example, memory unit (5042) of surgical instrument (5040) may store data related to what geographical location instrument (5040) was sold and sent to. Memory unit (5042) may include data containing the serial number identifying the specific instrument (5040). Surgical instrument (5040) may also include a QR code (5048). QR code (5048) may contain suitable data required to identify the specific instrument (5040) along with various suitable information related to the specific instrument (5040) (such as a geographical location in which instrument (5040) was sold and/or sent).

Instrument (5040) also includes a communication module (5046) in communication with processor (5044). Communication module (5046) is configured to establish communication with a corresponding communication module (5056) of disposal assistance device (5050) such that data may be shared between instrument (5040) and disposal assistance device (5050). Communication module (5046) may include any suitable communication means as would be apparent to one skilled in the art in view of the teachings herein. For example, communication module (5046) may be configured to communicate via Bluetooth technology, Near Field Communication “NCF”, Radio Frequency Identification “RFID”, etc.

Disposal assistance device (5050) may take any suitable form, such as a smart phone, a tablet, etc. Disposal assistance device (5050) may be used within the sterile field (5006) during disassembly of surgical subcomponents (5022) in accordance with the description herein. Additionally, or alternatively, disposal assistance device (5050) may be used outside the sterile field (5006) during reprocessing, remanufacturing, and/or disposal of surgical subcomponents (5022) in accordance with the description herein.

Disposal assistance device (5050) includes a memory unit (5052) and a processing unit (5054); which may together function as a control unit for disposal assistance device (5050). Memory unit (5052) may include a suitable application (e.g., software product) that includes download and uploading capabilities which may be used to access cleaning and sterilization protocols in accordance with the description herein. Disposal assistance device (5050) includes corresponding communication module (5056) configured to communicate with communication module (5046) described above. Communication module (5056) may include any suitable communication means as would be apparent to one skilled in the art in view of the teachings herein. For example, communication module (5056) may be configured to communicate via Bluetooth technology, Near Field Communication “NCF”, Radio Frequency Identification “RFID”, etc.

Disposal assistance device (5050) also includes a display (5055) in communication with processing unit (5054). Processing unit (5054) may instruct display (5055) to show suitable information to a user, such as information provided by running suitable applications described herein. Display (5055) may take any suitable form as would be apparent to one skilled in the art in view of the teachings herein. For example, display (5055) may be a touch screen. Disposal assistance device (5050) also includes a camera (5058) in communication with processing unit (5054). Camera (5058) may be utilized to capture and communicate images to processing unit (5054) such that suitable applications may utilize images in accordance with the description herein. Camera (5058) may include any suitable components as would be apparent to one skilled in the art in view of the teachings herein.

In some instances, the application running disposal assistance device (5050) may be configured to connect to a hub (106) that was used in conjunction with instrument (5040) during a surgical procedure. Such an application may have limited permissions with hub (106) such that the staff utilizing disposal assistance device (5050) and the corresponding application do not have access to any portion of hub (106) aside from relevant disposal instructions, cleaning, and/or sterilization information. Such connection between hub (106) and the application of device (5050) may allow device (5050) to communicate with interconnected hospital systems for operating room system/inventory tracking, etc. Therefore, once instrument (5040) or other suitable instruments/components are used in a surgery and scanned by device (5050), hub (106) may update the inventory of the hospital system to recognize use of instruments (5040) and/or other components. It should be understood that being “scanned” by device (5050) may include device (5050) utilizing camera (5056) to scan QR code (5048); establishing a connection between communication modules (5046, 5056); or any other suitable form of device (5050) obtaining data from instrument (5040) as would be apparent to one skilled in the art in view of the teachings herein.

In instances where disposal assistance device (5050) is utilized within surgical theater (5000), the application being utilized by disposal assistance device (5050) may communicate with a sterilization group located outside surgical theater (5000). Therefore, as disposal assistance device (5050) scans instrument (5040) and/or various subcomponents of instrument (5040) in order to obtain suitable reprocessing/disposal instructions in accordance with the description herein, the application running on device (5050) may inform the sterilization group that suitable portions of instrument (5040) intended for sterilization will be transported to the sterilization group.

Similarly, the application being utilized by disposal assistance device (5050) may communicate with a maintenance group to notify the maintenance group when equipment is ready for service. Therefore, as disposal assistance device (5050) scans instrument (5040) and/or various subcomponents of instrument (5040) in order to obtain suitable reprocessing/disposal instructions in accordance with the description herein, the application running on device (5050) may inform the maintenance group that suitable portions of instrument (5040) intended for maintenance are ready for service.

In instances where disposal assistance device (5050) is in communication with hub (106), disposal assistance device (5050) and hub (106) may be configured to confirm any lost or missing devices, such as instrument (5040), used during a procedure. For example, hub (106) may be configured to track what instruments (5040) or other devices are used during a procedure. During the disposal process, instruments (5040) used in the procedure may be scanned or otherwise processed utilizing disposal assistance device (5050). Communication between device (5050) and hub (106) may allow disposal assistance device (5050) and/or hub (106) to compare which instruments (5040) were used during a procedure and which instruments (5040) were scanned for suitable reprocessing/disposal. This comparison allows device (5050) and/or hub (106) to ensure all devices have been accounted for. In instances where a smart disposal system is used, disposal assistance device (5050) may confirm proper disposal via communication with a smart disposal system.

In some instances, the application running on disposal assistance device (5050), may connect with either remote server (113) and/or cloud (104) instead of hub (106). As one example, some hospital systems may not have access to a hub (106). In such instances, cloud (104) and/or remote service (113) may coordinate with the suitable application on device (5050) to provide cleaning, sterilization, and/or disposal protocols and provide step-by-step instruction in accordance with the teachings herein. In examples where application connects with either remote server (113) and/or cloud (104), such an application may auto-connect disposal assistance device (5050) with a suitable manufacturer call center for help.

In some instances, the application running on device (5050) may utilize location information to determine the country and/or region in which device (5050) is being used. For example, the application running on device (5050) may have application location services that determine the location of use by utilizing any suitable means as would be apparent to one skilled in the art in view of the teachings herein. In some instances, the application running on device (5050) may request the user to input a location of use when setting up the application account on device (5050).

In some instances, instrument (5040) may not be connected to hub (106) during a surgical procedure such that hub (106) does not collect performance data of surgical instrument (5040) during exemplary use. As mentioned above, memory (5042) of instruments (5040) may be operable to store various information related to the performance of surgical instrument (5040) during an exemplary surgical procedure in accordance with the description herein. For example, such information may include device motor data, sensor data, failures experienced by instrument (5040) during use, error codes, etc. In some instances, the application running on device (5050) may be configured to extract performance data stored on memory (5042) of instrument (5040) via communication modules (5046, 5056). Application running on device (5050) may then be configured to upload the extracted performance data onto hub (106), cloud (104), remote server (113), etc. In instances where no hub (106) is used, a gateway to device (5050) may be utilized to extract performance data. This extraction and uploading of data could be invisible to the operator utilizing device (5050) and could occur automatically once device (5050) establishes communication with instrument (5040) via communication modules (5046, 5056).

FIG. 8 shows a determination procedure (5060) that is configured to allow instrument (5040), generator module (140), and/or hub (106) to identify relevant factors of instrument (5040) usage and then access or determine a suitable disposal methodology based on the identified relevant factors. Determination procedure (5060) may be utilized with any suitable equipment as would be apparent to one skilled in the art in view of the teachings herein. For example, determination procedure (5060) may be utilized in conjunction with instrument (5040) and/or disposal assistance device (5050) described above. As another example, determination procedure (5060) may be utilized in conjunction with instrument (5040) and hub (106). As yet another example, determination procedure (5060) may be utilized in conjunction with instrument (5040) hub (106), and disposal assistance device (5050). While determination procedure (5060) is shown and described in a particular order of steps, it should be understood that some steps may be entirely optional. Further, it should be understood that the order in which steps are performed may have any suitable order as would be apparent to one skilled in the art in view of the teachings herein.

As mentioned above, standards and/or guidelines for disposal methodology may deviate depending on the location which instrument (5040) is used. Therefore, one step (5062) in the determination procedure (5060) may include determining the location of the surgical theater (5000) in which the procedure is being performed. Any suitable components or means may be utilized in order to determine the location of the surgical theater (5000) as would be apparent to one skilled in the art in view of the teachings herein. For example, the application running on disposal assistance device (5050) may be configured to determine the country and/or region in which device (5050) is being used; which may then be used to infer the location of the surgical theater (5000).

As another example, instrument (5040) and/or generator module (140) may have a device code and/or serial number that contains or provides access to the specified location to which instrument (5040) was shipped to for intended use in accordance with the description herein. In instances where the device code and/or serial number is associated with instrument (5040), when instrument (5040) couples with generator module (140)/hub (106)/cloud (104), generator module (140)/hub (106)/cloud (104) may read the device code provided by instrument (5040) in order to determine the location of intended use. In some instances, generator (140) may be pre-programmed by the manufacturer with the intended location in which generator (140) is to be shipped; such as utilizing program EEPROM code. In such instances, generator (140) may determine the location of use by referencing this pre-programmed information during exemplary use. Further, when generator (140) is activated, generator (140) may perform an initialization check in order to identify location of use. Such location of use may be stored in the field service settings.

As another example, rather than a device code and/or serial number, generator module (140) and/or instrument (5040) may include another identifier, such and an RFID chip that would be able to track the sale of the product to determine its location of intended use. In some instances, the location of intended use, as determined by the manufacturer, may be cross checked with a suitable means of identifying actual use (some examples of identifying actual use are provided below). In instances where the location of intended use does not match with the identified location of actual use, hub (106) and/or cloud (104) may be notified to allow special processing of the device tracking by the manufacturer.

As another example, a GPS device may be associated with instrument (5040), device (5050), hub (106), generator module (140), or any other suitable device that would be apparent to one skilled in the art in view of the teachings herein. Once activated, the GPS device may determine the location of use and communicate that information to any suitable component, such as hub (106), generator module (140), device (5050), and/or instrument (5040). As another example, hub (106) and/or device (5050) may analyze the network in which they are connected to in order to determine the location of use. As another example, generator, hub (106) and/or device (5050) may have a hospital identifier that may determine the location of use. As another example, hub (106) and/or device (5050) may determine location of use by connecting to remote server (113), and/or cloud (104). After connecting to the remote server (113) and/or cloud (104), hub (106) and/or device (5050) may access manufacturer-generated regional codes that specify a location of use. As another example, hub (106), device (5050), and/or any other suitable component of surgical system (100) may provide an interface allowing a user to select their region of use. As another example, hub (106) and/or device (5050) may utilize its associated IP address to determine the location of use. As another example, hub (106) and/or device (5050) may utilize its software license to determine the location of use.

After suitably determining the location of use (5062), instrument (5040), hub (106), and/or device (5050) may then provide location information (5064) to the suitable applications or devices that are used in determining and/or accessing the suitable disposal methodology. The provided location information (5064) may be utilized by disposal assistance device (5050), remote server (113), cloud (104) and/or hub (106). It should be understood that providing location information (5064) may occur at any suitable time during determination procedure (5060) as would be apparent to one skilled in the art in view of the teachings herein. For example, providing location information (5064) may occur simultaneously with other steps in determination procedure (5060). Information may be provided automatically or in response to a specific action; such as device (5050) scanning instrument (5040) or communicating with instrument (5040) via communication modules (5046, 5056).

During a surgical procedure in accordance with the description herein, instrument (5040) and/or hub (106) may track if any other products were used in combination with instrument (5040). For example, instrument (5040) and/or hub (106) may track if any drugs, controlled substances, adjuncts, or any other suitable medical products as would be apparent to one skilled in the art in view of the teachings herein. Use of such medical products may affect the standards and/or guidelines for disposal methodology used for processing instrument (5040) after exemplary use. Therefore, one step (5066) in the determination procedure (5060) may include providing information regarding the various relevant medical products used in combination with instrument (5040). Instrument (5040), hub (106), and/or device (5050) may then provide such medical product information (5066) to the suitable applications or devices that are used in determining and/or accessing the suitable disposal methodology. For example, the provided medical product information (5066) may be utilized by disposal assistance device (5050), remote server (113), cloud (104) and/or hub (106). Information may be provided automatically or in response to a specific action; such as device (5050) scanning instrument (5040) or communicating with instrument (5040) via communication modules (5046, 5056).

While in the current example, location information (5064) and relevant medical product information (5066) is provided to help determine standards and/or guidelines for the disposal methodology in some instances just the location information (5064), or just the medical product information (5066) may be utilized to help determine standards and/or guidelines for disposal methodology. Additionally, any other suitable information may also be provided as well, in combination with the other parameters or alone. For example, performance data accumulated during use of surgical instrument (5040) that is accessed (5070) and uploaded (5072) in accordance with the description herein may be provided to the suitable applications or devices that are used in determining and/or accessing the suitable disposal methodology.

Hub (106) and/or disposal assistance device (5050) may access a selective lookup table (5068) that includes the various local disposal methodologies. Utilizing the suitable information provided (5064, 5066, 5070, 5072), hub (106) and/or disposal assistance device (5050) selects the suitable local disposal methodology from the various methodologies provided on the selective lookup table. The various standards and/or guidelines for local disposal methodology may be stored on any suitable device as would be apparent to one skilled in the art in view of the teachings herein. For example, lookup tables may be stored on cloud (104), remote server (113), hub (106), disposal assistance device (5050), etc. Accessing the selective lookup table (5068) may occur automatically or in response to a specific action; such as device (5050) scanning instrument (5040) or communicating with instrument (5040) via communication modules (5046, 5056).

As mentioned above, instrument (5040) may store performance data on memory (5042). In some examples, as also mentioned above, when instrument (5040) is scanned by disposal assistance device (5050) or communication between instrument (5040) and device (5050) is established via communication module (5046, 5056), such information may be accessed (5070) and uploaded (5072) to cloud (104). Cloud (104) may store uploaded performance data for various purposes. In some instances, uploading performance data to cloud (104) via communication between instrument (5040) and device (5050) may be beneficial when a hub (106) is not utilized during the surgical procedure.

With the proper standards and/or guidelines for local disposal methodology accessed, determination procedure (5060) may then display (5074) the local disposal procedure to the staff in charge of performing the disposal methodology. The disposal procedure may include displaying chronological instructions of the disposal methodology. Such instructions may include how to disassemble instrument (5040); what components of instrument (5040) are reusable and which components of instrument (5040) are disposable; how to properly prepare such components for disposal or reuse; how to properly store such components for disposal or reuse; etc. Such a display (5074) may be shown on device (5050) or suitable components of hub (106). Display (5074) may allow a user to scroll through the chronological instructions of the disposal methodology and indicate when each instruction is accomplished.

While the staff performs the disposal methodology in accordance with the displayed standards and/or guidelines displayed, hub (106) and/or device (5050) may notify sterilization team of incoming device (5078) expected to be transported to the sterilization team in light of the displayed disposal methodology. It should be understood that in instances where the sterilization staff is utilizing determination procedure (5060), such a notification (5078) may be omitted.

### IV. Exemplary Generator Storage Bin with Reusable Cord and Sterilization Means

In some instances, surgical kit (5020) described above comes with a power cord configured to electrically couple surgical subcomponents (5022) to generator module (140) such that generator module (140) may electrically power surgical subcomponents (5022) during exemplary use in accordance with the description herein. Cords may also enable data to communicate between surgical subcomponents (5022) and hub (106). In some instances, it may be desirable to have at least a portion of cord be reusable with generator module (140), rather than be associated with surgical kit (5020). Therefore, after a procedure, the portion of cord associated with generator module (140) may remain associated with generator module (140), and the portion of cord associated with surgical kit (5020) may be categorized as a post-surgery component (5032, 5034, 5036) for reuse or disposal as would be apparent to one skilled in the art in view of the teachings herein.

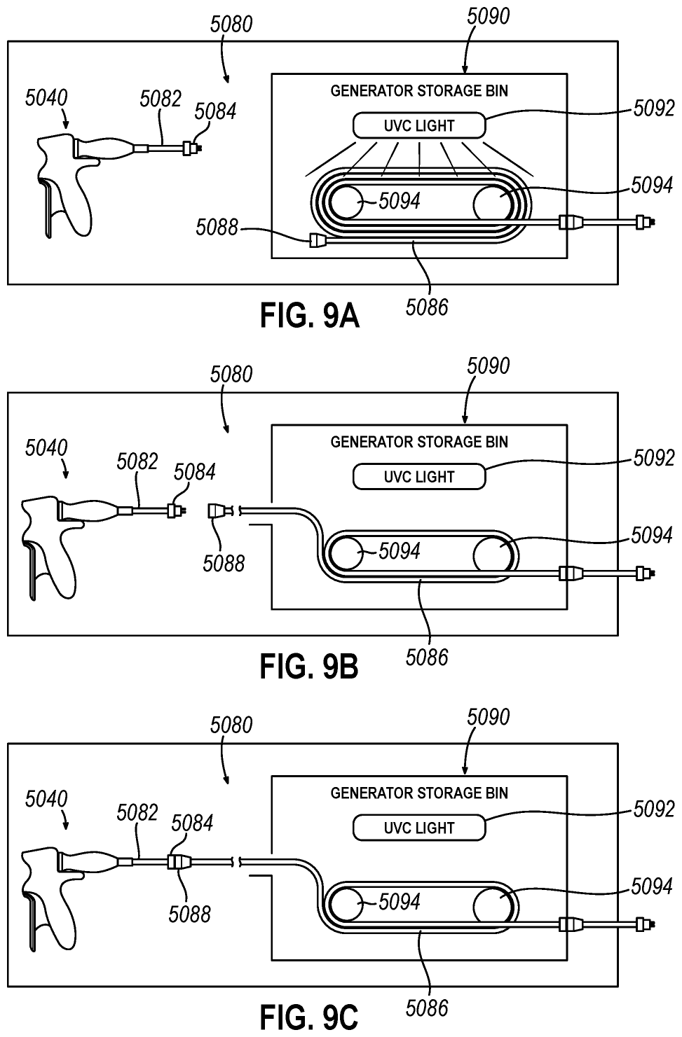

FIGS. 9A-9C show an exemplary power coupling assembly (5080) and generator storage bin (5090) that may be readily incorporated into surgical kit (5020) and generator module (140). Power coupling assembly (5080) includes a short cord (5082) and a long cord (5086). Short cord (5086) may be associated with instrument (5040) formed by surgical kit (5020) such that short cord (5082) comes packaged with surgical kit (5020). Short cord (5082) contains a coupling member (5084) configured to selectively establish communication with long cord (5086) and generator module (140) via coupling member of long cord (5088). When short cord (5082) is coupled with long cord (5086), generator module (140) may actively power instrument (5040) in accordance with the description herein.

Long cord (5086) is attached to generator module (140) and is wrapped and stored internally within generator storage bin (5090) of generator module (140) via couplings posts (5094). Since long cord (5086) remains associated with generator module (140) and is configured to couple with short cord (5082), long cord (5086) may reduce the amount of disposal from surgical kit (5020). In some instances, no short cord (5082) is present such that long cord (5086) is coupled directly with instrument (5040). In some instances, device contains a detachable, reusable power cord with a connection junction positioned at the end of handpiece. The connection can be severed with a physical unique key that may require multiple actions. The connection can alternatively be severed by an electronically activated release mechanism that could be initiated via the user or the generator module (140).

Storage bin (5090) may include a pop top with a bin for cord storage and multi-tool storage. Rather than posts (5094), storage bin (5090) may have a hook with cord (5086) wrapped around hook.

Storage bin (5090) includes a UV light (5092). UV light (5092) may be utilized to sterilize long cord (5086) after exemplary use and while long cord (5086) is stored within storage bin (5090). FIGS. 9A-9C show an exemplary coupling of cords (5082, 5086). First, as shown in FIG. 9A, long cord (5086) and coupling member (5088) is within storage bin (5090). Next, coupling member (5088) may be removed from storage bin (5090), as shown in FIG. 9B. Finally, coupling members (5084, 5088) may be coupled to power instrument (5040) as shown in FIG. 9C.

### V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of determining a disposal methodology of a surgical kit, the method comprising: (a) determining a geographical location in which the surgical kit is being used; (b) generating a location data set based on the geographical location in which the surgical kit is being used; (c) providing the location data set to a resource device; (d) receiving the disposal methodology from the resource device based on the location data set; and (e) displaying a set of instructions based on the disposal methodology received from the resource device.

### Example 2

The method of any one or more of the previous Examples, wherein the resource device comprises a cloud storage system.

### Example 3

The method of any one or more of the previous Examples, wherein the cloud storage system comprises a plurality of disposal methodologies each directed to a different geographical location.

### Example 4

The method of any one or more of the previous Examples, further comprising determining a list of medical products used in conjunction with the surgical kit.

### Example 5

The method of any one or more of the previous Examples, further comprising generating a medical product data set based on the list of medical products used in conjunction with the surgical kit.

### Example 6

The method of any one or more of the previous Examples, further comprising providing the medical product data set to the resource device.

### Example 7

The method of any one or more of the previous Examples, further comprising receiving the disposal methodology from the resource device based on the medical product data set.

### Example 8

The method of any one or more of the previous Examples, further comprising displaying the set of instructions on a disposal assistance device.

### Example 9

The method of any one or more of the previous Examples, further comprising displaying the set of instructions on a display screen of a surgical hub configured to be used in conjunction with the surgical kit.

### Example 10

The method of any one or more of the previous Examples, wherein determining the geographical location further comprises using a serial code on the surgical kit.

### Example 11

The method of any one or more of the previous Examples, wherein determining the geographical location further comprises reading a pre-programed code on a generator module.

### Example 12

The method of any one or more of the previous Examples, wherein determining the geographical location further comprises receiving a use input.

### Example 13

The method of any one or more of the previous Examples, wherein determining the geographical location further comprises utilizing a network address.

### Example 14

The method of any one or more of the previous Examples, wherein the surgical kit comprising a hand held device.

### Example 15

The method of any one or more of the previous Examples, wherein the surgical kit comprises a battery.

### Example 16

The method of any one or more of the previous Examples, wherein the surgical kit comprises an ultrasonic transducer.

### Example 17

The method of any one or more of the previous Examples, wherein the surgical kit comprises a power cord.

### Example 18

A method of determining a disposal methodology of a surgical kit, the method comprising: (a) identifying a geographical location of use for the surgical kit; (b) providing the geographical location of use to a resource device storing a plurality of disposal methodologies based; (c) receiving a particular disposal methodology from the resource device based at least in part on the geographical location provided to the resource device; and (d) displaying a set of instructions based on the particular disposal methodology received from the resource device.

### Example 19

The method of any one or more of the previous Examples, wherein the set of instructions are arranged in a chronological order.

### Example 20

A surgical system comprising: (a) a generator; (b) a surgical kit configured to be assembled into a surgical instrument, wherein the surgical instrument is configured to be disassembled into a first set of used components and a second set of used components; (c) an identification feature associated with either the generator of the surgical kit; and (d) a disassembly assistant device configured to display a set of instructions on how to disassemble the surgical instrument into the first set of used components and the second set of used components, wherein the set of instructions is dependent on data obtained from the identification feature.

### Example 21

A method of determining a disposal methodology of a surgical kit, the method comprising: (a) identifying a geographical location of use for the surgical kit; (b) extracting a set of disposal instructions from a lookup table based on the identified geographical location of use for the surgical kit; and (c) displaying the disposal instructions.

### VI. Miscellaneous

Versions of the devices described above may have application in conventional medical treatments and procedures conducted by a medical professional, as well as application in robotic-assisted medical treatments and procedures.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

In addition to the foregoing, the teachings herein may be readily combined with the teachings of U.S. Pat. App. No. [ATTORNEY DOCKET NO. END9447USNP1.0754992], entitled “Method of Reclaiming Portions of Surgical Instruments for Remanufacturing and Sustainability,” filed on even date herewith, the disclosure of which is incorporated by reference herein. Various suitable ways in which the teachings herein may be combined with the teachings of U.S. Pat. App. No. [ATTORNEY DOCKET NO. END9447USNP1.0754992] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP1.0754994], entitled “Surgical Instrument with Predetermined Separation Features for Waste Stream Utilization and Related Methods,” filed on even date herewith, the disclosure of which is incorporated by reference herein. Various suitable ways in which the teachings herein may be combined with the teachings of U.S. Pat. App. No. [ATTORNEY DOCKET NO. END9448USNP1.0754994] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP2.0754977], entitled “Surgical Instrument with Removable Cable and Associated Couplings,” filed on even date herewith, the disclosure of which is incorporated by reference herein. Various suitable ways in which the teachings herein may be combined with the teachings of U.S. Pat. App. No. [ATTORNEY DOCKET NO. END9448USNP2.0754977] will be apparent to those of ordinary skill in the art in view of the teachings herein.

In addition to the foregoing, the teachings herein may be readily combined with the teachings of U.S. Pat. App. No. [ATTORNEY DOCKET NO. END9448USNP3.0754979], entitled “Surgical System and Methods of Assembly and Disassembly of Surgical Instrument,” filed on even date herewith, the disclosure of which is incorporated by reference herein. Various suitable ways in which the teachings herein may be combined with the teachings of U.S. Pat. App. No. [ATTORNEY DOCKET NO. END9448USNP3.0754979] will be apparent to those of ordinary skill in the art in view of the teachings herein.

In addition to the foregoing, the teachings herein may be readily combined with the teachings of U.S. Pat. App. No. [ATTORNEY DOCKET NO. END9449USNP1.0754981], entitled “Robotic Surgical System with Removable Portion and Method of Disassembling Same,” filed on even date herewith, the disclosure of which is incorporated by reference herein. Various suitable ways in which the teachings herein may be combined with the teachings of U.S. Pat. App. No. [ATTORNEY DOCKET NO. END9449USNP1.0754981] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP2.0754999], entitled “Reclamation Packaging for Surgical Instrument and Related Methods,” filed on even date herewith, the disclosure of which is incorporated by reference herein. Various suitable ways in which the teachings herein may be combined with the teachings of U.S. Pat. App. No. [ATTORNEY DOCKET NO. END9450USNP2.0754999] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP3.0755001], entitled “Surgical Instrument with Various Alignment Features and Methods for Improved Disassembly and Assembly,” filed on even date herewith, the disclosure of which is incorporated by reference herein. Various suitable ways in which the teachings herein may be combined with the teachings of U.S. Pat. App. No. [ATTORNEY DOCKET NO. END9450USNP3.0755001] will be apparent to those of ordinary skill in the art in view of the teachings herein.

In addition to the foregoing, the teachings herein may be readily combined with the teachings of U.S. Pat. App. No. [ATTORNEY DOCKET NO. END9450USNP4.0755006], entitled “Surgical System and Methods for Instrument Assessment and Cleaning,” filed on even date herewith, the disclosure of which is incorporated by reference herein. Various suitable ways in which the teachings herein may be combined with the teachings of U.S. Pat. App. No. [ATTORNEY DOCKET NO. END9450USNP4.0755006] will be apparent to those of ordinary skill in the art in view of the teachings herein.

It should also be understood that any ranges of values referred to herein should be read to include the upper and lower boundaries of such ranges. For instance, a range expressed as ranging “between approximately 1.0 inches and approximately 1.5 inches” should be read to include approximately 1.0 inches and approximately 1.5 inches, in addition to including the values between those upper and lower boundaries.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of determining a disposal methodology of a surgical kit, the method comprising:
(a) determining a geographical location in which the surgical kit is being used;
(b) generating a location data set based on the geographical location in which the surgical kit is being used;
(c) providing the location data set to a resource device;
(d) receiving the disposal methodology from the resource device based on the location data set; and
(e) displaying a set of instructions based on the disposal methodology received from the resource device.

2. The method of claim 1, wherein the resource device comprises a cloud storage system.

3. The method of claim 2, wherein the cloud storage system comprises a plurality of disposal methodologies each directed to a different geographical location.

4. The method of claim 1, further comprising determining a list of medical products used in conjunction with the surgical kit.

5. The method of claim 4, further comprising generating a medical product data set based on the list of medical products used in conjunction with the surgical kit.

6. The method of claim 5, further comprising providing the medical product data set to the resource device.

7. The method of claim 6, further comprising receiving the disposal methodology from the resource device based on the medical product data set.

8. The method of claim 1, further comprising displaying the set of instructions on a disposal assistance device.

9. The method of claim 1, further comprising displaying the set of instructions on a display screen of a surgical hub configured to be used in conjunction with the surgical kit.

10. The method of claim 1, wherein determining the geographical location further comprises using a serial code on the surgical kit.

11. The method of claim 1, wherein determining the geographical location further comprises reading a pre-programed code on a generator module.

12. The method of claim 1, wherein determining the geographical location further comprises receiving a use input.

13. The method of claim 1, wherein determining the geographical location further comprises utilizing a network address.

14. The method of claim 1, wherein the surgical kit comprising a hand held device.

15. The method of claim 1, wherein the surgical kit comprises a battery.

16. The method of claim 1, wherein the surgical kit comprises an ultrasonic transducer.

17. The method of claim 1, wherein the surgical kit comprises a power cord.

18. A method of determining a disposal methodology of a surgical kit, the method comprising:
(a) identifying a geographical location of use for the surgical kit;
(b) providing the geographical location of use to a resource device storing a plurality of disposal methodologies based;
(c) receiving a particular disposal methodology from the resource device based at least in part on the geographical location provided to the resource device; and
(d) displaying a set of instructions based on the particular disposal methodology received from the resource device.

19. The method of claim 18, wherein the set of instructions are arranged in a chronological order.

20. A surgical system comprising:
(a) a generator;
(b) a surgical kit configured to be assembled into a surgical instrument, wherein the surgical instrument is configured to be disassembled into a first set of used components and a second set of used components;
(c) an identification feature associated with either the generator of the surgical kit; and
(d) a disassembly assistant device configured to display a set of instructions on how to disassemble the surgical instrument into the first set of used components and the second set of used components, wherein the set of instructions is dependent on data obtained from the identification feature.
